(19) United States
(12) Patent Application Publication
(10) Pub. No.: US 2009/0036875 A1
(43) Pub. Date: Feb. 5, 2009

(54) CARDIAC TISSUE THERAPY

(76) Inventors: ROBERT GLENMORE WALSH, Lakeville, MN (US); Jin Shimada, Grantsburg, WI (US)

Correspondence Address: MERCHANT & GOULD PC, P.O. BOX 2903, MINNEAPOLIS, MN, 55402-0903, US

(21) Appl. No.: 11/830,393

(22) Filed: Jul. 30, 2007

(51) Int. Cl. A61K 9/22

(52) U.S. Cl. 604/890.1

(57) ABSTRACT

A method and apparatus for treating tissue of a heart of a patient includes delivering a carrier to an epicardial surface of the heart. The carrier has a therapeutic agent selected for treating cardiac tissue. The agent is releasable from the carrier over a period of time following placement of the carrier at the epicardial surface of the heart. The carrier is contained within a delivery tool at a first geometry and changes to a second geometry following delivery of the carrier from the tool.

### I. CROSS-REFERENCE TO RELATED APPLICATION

This application claims subject matter disclosed in commonly assigned and co-pending U.S. patent application Ser. No. [not yet assigned] filed on even date herewith and in the name of the same inventors as the present application and titled “Conjunctive Stent Therapy”.

### II. BACKGROUND OF THE INVENTION

1. Field of the Invention

This invention pertains to treating cardiac tissue. More particularly, this invention pertains to such a treatment a therapy including placement of a therapeutic agent applied at an epicardial surface of a patient's heart.

2. Description of the Prior Art

a. Coronary Artery Disease Treatments

The history of treatment of coronary artery disease includes a progressive development of less-invasive procedures for treating coronary vessel occlusion. Traditional bypass procedures include harvesting a patient's blood vessel and using the harvested vessel to create a new blood flow path which terminates distal to a coronary occlusion. Historically, such surgical procedures required a highly invasive sternotomy. Less invasive surgical procedures such as thoracotomies have been developed.

Non-surgical (i.e., percutaneous) options have been developed for treating occluded blood vessels. Angioplasty involves placement of a balloon in a coronary vessel at an occlusion site. The balloon is inflated to improve the patency of the blood vessel. Further, mechanical supports (i.e., stents) have been developed for placement in the blood vessel at the occlusion site. Such stents may be self-expanding or balloon expanded.

Historically, coronary stents were bare metal stents (e.g., stainless steel, nitinol or other bio-compatible material). More recently, drug-eluting stents have been developed. These stents include an anti-restenosis drug to abate restenosis following placement of the stent. Commonly, the drug is carried in a polymer matrix permitting delayed release of the drug over a period of time following stent placement.

Drug-eluting stents have exhibited a material improvement in abating restenosis. However, with passage of time, such stents have exhibited an apparent increased likelihood of thrombosis relative to bare metal stents. It is believed the thrombogenic experience is related to the polymer matrix which carries the anti-restenosis drug. Namely, after the anti-restenosis drug is discharged from the matrix, the polymer of the matrix remains on the stent and presents a site for thrombus formation.

One of several inventions disclosed in this application includes a conjunctive therapy of a stent and an epicardial delivered anti-restenosis agent.

b. Pericardial Access Procedures

The pericardium is a sack surrounding the exterior surface (i.e., epicardium or epicardial surface) of the heart. Incisions through the pericardium can result in adhesions which may complicate future heart treatments.

Less invasive procedures have been described for accessing the pericardial space (i.e., the space defined between the pericardium and the epicardial surface). These include surgical and percutaneous procedures. As used herein, “surgical” access means accessing a pericardial space from an exterior side of the pericardium. “Percutaneous” access means accessing the pericardial space without penetrating through the tissue of the pericardium.

Examples of such less invasive surgical procedures are shown in the following (all of which are incorporated herein by reference as though set forth in full): U.S. Pat. No. 5,634,895 to Igo et al. issued Jun. 3, 1997; U.S. patent application Publication No. US 2006/0074373 to Walsh et al. published Apr. 6, 2006; U.S. patent application Publication No. US 2006/0189840 to Walsh et al. published Aug. 24, 2006; U.S. patent application Publication No. US 2005/0261673 to Bonner et al. published Nov. 24, 2005 (also enumerating various therapeutic agents); U.S. Pat. No. 7,186,214 to Ness issued Mar. 6, 2007; U.S. Pat. No. 6,206,004 to Schmidt et al. issued Mar. 27, 2001; U.S. Pat. No. 6,156,009 to Grabek issued Dec. 5, 2004; U.S. Pat. No. 5,972,013 to Schmidt issued Oct. 26, 1999 and U.S. Pat. No. 5,931,810 to Grabek issued Aug. 3, 1999.

Examples of such less invasive percutaneous procedures are shown in the following (all of which are incorporated herein by reference as though set forth in full): U.S. Pat. No. 5,269,326 to Verrier issued Dec. 14, 1993; U.S. Pat. No. 6,200,303 to Verrier et al. issued Mar. 13, 2001; U.S. patent application Publication No. US 2001/0039410 to Verrier et al. published Nov. 8, 2001; U.S. Pat. No. 5,968,010 to Waxman et al. issued Oct. 19, 1999; U.S. Pat. No. 6,582,536 to Shimada issued Jun. 24, 2003; U.S. Pat. No. 7,207,988 to Leckrone et al. issued Apr. 24, 2007; U.S. Pat. No. 6,692,458 to Forman et al. issued Feb. 17, 2004; U.S. Pat. No. 4,884,567 to Elliott et al. issued Dec. 5, 1989; U.S. Pat. No. 6,613,062 to Leckrone et al. issued Sep. 2, 2003; U.S. patent application Publication No. US 2006/0247672 to Vidlund et al. published Nov. 2, 2006; U.S. patent application Publication No. US 2007/0010793 to Callas et al. published Jan. 11, 2007; U.S. patent application Publication No. US 2006/0173441 to Gelfand et al. published Aug. 3, 2006; U.S. patent application Publication No. US 2006/0074397 to Shimada published Apr. 6, 2006; and U.S. Pat. No. 5,087,243 to Avitall issued Feb. 11, 1992 (describing myocardial iontophoresis).

One of several inventions disclosed in this application includes a novel method and apparatus for delivery of a therapeutic agent to the pericardial space.

c. Therapeutic Agents

The description of the present invention includes placement of a cardiac therapeutic agent at an epicardial surface. Such agents are well-known in the art and form no part of this invention per se. Examples of such are described in the following (all of which are incorporated herein by reference as though set forth in full): U.S. patent application Publication No. US 2003/0060415 to Hung published Mar. 27, 2003 (enumerating a variety of such agents including anti-restenosis agents and agents administered to the epicardial space); U.S. patent application Publication No. US 2005/0261673 to Bonner et al. published Nov. 24, 2005; U.S. Pat. No. 5,634,895 to Igo et al. issued Jun. 3, 1997 (including describing delivery of anti-restenosis agents to the pericardial space); U.S. patent application Publication No. US 2003/0032998 to Altman published Feb. 13, 2003 (including describing a rolled epicardial patch for distributing a drug to an epicardial surface—e.g., paragraph 0084 of the '998 application); U.S. Pat. No. 6,977,080 to Donovan issued Dec. 20, 2005; International Publication No. WO 2006/076342 A2 published Jul. 20, 2006; U.S. patent application Publication No. US 2003/0109442 to Bisgaier et al. published Jun. 12, 2003 (describing restenosis treatment with localized delivery of therapeutic agent); U.S. patent application Publication No. US 2004/0102759 to Altman et al. published May 27, 2004 (including treating of coronary artery disease with therapeutic agents injected into heart wall tissue); U.S. patent application Publication No. US 2006/0084943 to Rosenman et al. published Apr. 20, 2006 (including therapeutic agent delivery carriers implanted into heart wall tissue); U.S. patent application Publication No. US 2006/0292125 to Kellar et al. published Dec. 28, 2006; U.S. patent application Publication No. US 2007/0078620 to Seward et al. published Apr. 5, 2007 (including method and kits for delivering pharmaceutical agents to adventia surrounding a blood vessel); U.S. patent application Publication No. US 2003/0004141 to Brown published Jan. 2, 2003 (including describing polymeric compounds impregnated with a therapeutic agent for delayed delivery); U.S. patent application Publication No. 2003/0009145 to Struijker-Boudier et al. published Jan. 9, 2003 (including describing delivery of drugs from sustained release devices implanted in myocardial tissue or in the pericardial space); U.S. Pat. No. 6,333,347 to Hunter et al. issued Dec. 25, 2001 (describing intrapericardial delivery of agents for treating a variety of cardiac diseases); U.S. Pat. No. 5,482,925 to Keefer et al. issued Jul. 22, 1997 (describing nitric oxide releasing polymers to treat restenosis); U.S. Pat. No. 7,208,011 to Shanley et al. issued Apr. 24, 2007 (describing an implantable medical device with drug filled holes) and U.S. Pat. No. 5,387,419 to Levy et al. issued Feb. 7, 1995 (controlled release, site specific myocardial agents in polymeric matrix at epicardium).

### III. SUMMARY OF THE INVENTION

According to a preferred embodiment of the present invention, a method and apparatus are disclosed for treating tissue of a heart of a patient. The method comprises delivering a carrier to an epicardial surface of the heart. The carrier has a therapeutic agent selected for treating cardiac tissue. The agent is releasable from the carrier over a period of time following placement of the carrier at the epicardial surface of the heart. The carrier is contained within a delivery tool at a first geometry and changes to a second geometry following delivery of the carrier from the tool.

### IV. BRIEF DESCRIPTION OF THE DRAWINGS

FIG. 1 is a schematic side-sectional view showing a coronary artery at an epicardial surface of a heart and spaced from a pericardium and showing an occlusion within a coronary artery;

FIG. 2 is a cross-sectional view of FIG. 1;

FIG. 3 is the view of FIG. 1 showing an occlusion treated by a prior art balloon angioplasty;

FIG. 4 is the view of FIG. 1 showing the occlusion treated by a prior art placement of a stent within the coronary artery;

FIG. 5 is the view of FIG. 1 showing a coronary obstruction treated in combination with a device according to the present invention for use as an adjunctive therapy;

FIG. 6 is a top plan view of an epicardial surface of the heart showing the device of the present invention overlying a treated area of a coronary artery (shown in phantom lines);

FIG. 7 is the view of FIG. 5 showing treatment of the occlusion site with the device delivered through a fat layer;

FIG. 8 is a schematic, longitudinal cross-sectional view of a distal end of a deployment device with an implant of the present invention shown within the distal end in a first geometry and positioned to be ejected from the delivery device by a push rod;

FIG. 9 is the view of FIG. 8 following ejection of the implant from the delivery device and with the implant assuming a second geometry;

FIG. 10 is a view shown in exploded format of a kit according to the present invention;

FIG. 11 is a top plan view of an alternative embodiment of the present invention showing an implant at an epicardial surface (not separately shown) over a site of a coronary occlusion treated by a prior art stent and with the coronary artery shown in cross-section to reveal the stent;

FIGS. 12 through 15 are the view of FIG. 11 showing further alternative embodiments of the present invention;

FIG. 15A is a cross-sectional view of the implant of FIG. 15;

FIG. 16 is a side-sectional view of a coronary artery in a myocardium and showing (in perspective) an alternative embodiment of the present invention within heart tissue at a site of a coronary artery occlusion treatment; and

FIGS. 17-22 are the view of FIG. 16 showing still further embodiments of the present invention.

### V. DESCRIPTION OF THE PREFERRED EMBODIMENT

Referring now to the several drawing figures in which identical elements are numbered identically throughout, a description of a preferred embodiment of the present invention will now be provided. The present invention is described in a currently preferred embodiment as an adjunctive treatment with prior art coronary artery stents. However, the invention is not intended to be so limited and may include a stand-alone therapy for a wide variety of uses including treatments for ischemia, infarction, vulnerable plaque and other cardiac disorders.

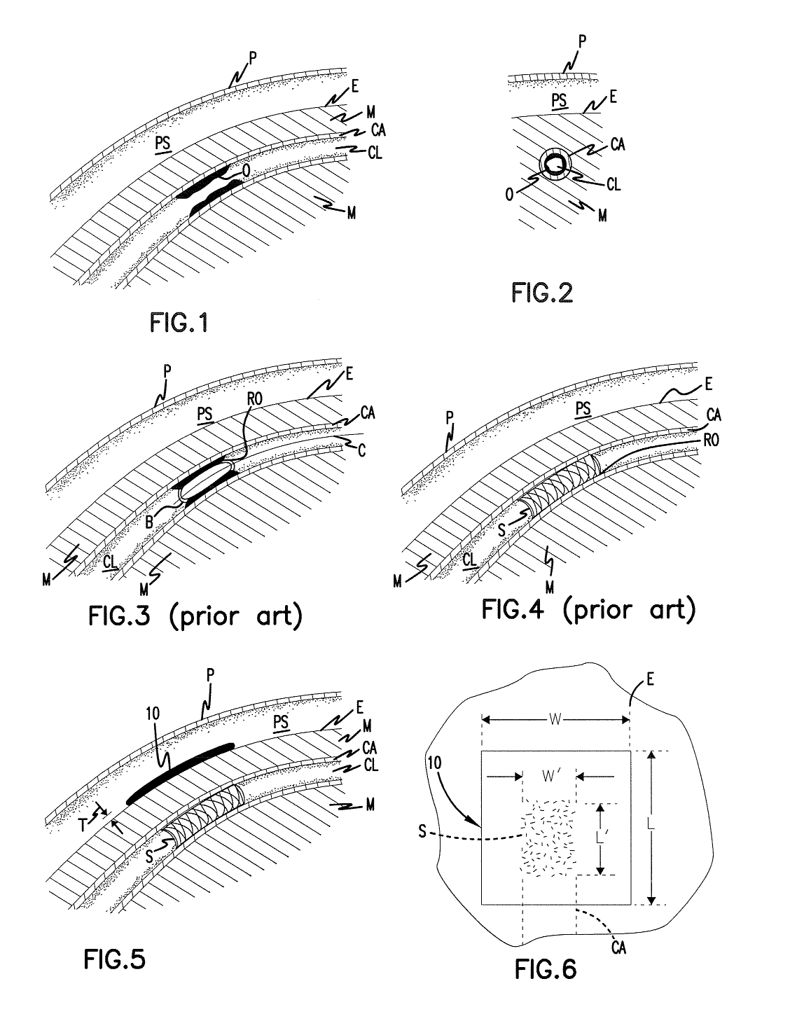

By way of background, FIGS. 1 and 2 illustrate, in schematic format, representative human anatomy. A portion of a patient's heart is shown as the heart wall or myocardium M. A coronary artery CA is shown imbedded within the myocardium M. The coronary artery CA defines a coronary lumen CL. The patient in FIGS. 1 and 2 suffers from coronary artery disease illustrated by an obstruction O contained within the coronary lumen CL which reduces blood flow through the coronary artery CA and which can lead to ischemia or infarction. The outer surface of the heart is the epicardium E. Overlying the epicardium E is the pericardium P. Opposing surfaces of the pericardium P and the epicardium E define the pericardial space PS.

FIGS. 3 and 4 illustrate prior art therapies for treating a patient with the obstruction O of FIGS. 1 and 2. FIG. 3 illustrates a balloon angioplasty procedure where a catheter C is inserted into the lumen of the coronary artery CA. The distal end of the catheter C contains an inflatable balloon B which is positioned at the obstruction O. The balloon B is inflated urging the obstruction O to compress against the walls of the coronary artery CA to present a reduced obstruction RO. The balloon is deflated and the catheter C and balloon B are removed. With the reduced obstruction RO, improved blood flow is attained through the coronary artery CA. FIG. 4 illustrates a prior art stenting procedure where a stent S is placed within the coronary artery CA at the site of the obstruction O. The stent S is expanded to reduce the obstruction. Unlike balloon angioplasty, the stent S remains in place at the site of the coronary artery disease. Stents come in many sizes and constructions. Stents may be bare metal stents fabricated from bio-compatible material as nitinol or stainless steel or the like.

With the prior art procedures of FIGS. 3 and 4, blood flow through the coronary artery is improved. However, the patient is at risk of restenosis whereby the site of the obstruction may, over time, become obstructed again. To reduce the likelihood of restenosis, stents S may be coated or impregnated with anti-restenosis drugs. These drugs are released from the stent S over time to prevent restenosis. The delayed delivery of a drug following initial placement of a stent S may occur over a period of, for example, 30-45 days. In this period of time, the drug reduces the inflammatory response and/or smooth muscle cell proliferation during a healing period.

While drug-eluting stents have been very effective in reducing restenosis, long-term (for example, two to four years following stent placement) drug-eluting stents are shown in certain studies to have higher incidences of thrombus formation over bare metal stents. While bare metal stents (i.e. not drug-eluting stents or drug-coated stents) are more likely to experience restenosis during a short-term period (e.g., less than 6 to 12 months), such stents which do not restenosis are more likely to have successful endothelial growth relative to drug-eluting stents and less potential for late term thrombosis (e.g., after one year). With such endothelial growth, blood flow to the coronary artery is not exposed directly to the metal of the stent thereby reducing the likelihood of platelet activation or other responses to foreign bodies within the blood flow. It is believed that such thrombus formation is due to the fact that the sites on the stent which release the drug present blood-contact surface which can produce thrombus formation.

In its most preferred embodiment, the present invention is an adjunct procedure for use with placement of a bare metal stent at an occlusion site within a coronary artery. The invention is illustrated in FIGS. 5 and 6 as an implant 10 in the form of a implant placed on the epicardial surface E overlying the site of a stent S. In FIG. 5, the coronary artery CA is shown imbedded within the myocardium M. It will be appreciated that coronary arteries may be superficial and located just at the epicardial surface E.

The implant 10 is shown in FIGS. 5 and 6 as a implant with a width W greater than a width W′ of the coronary artery CA and with a length L greater than a length L′ of the stent S. As a result, the implant 10 presents a surface area greater than the surface area of the stent at the epicardial surface E so that the entire area of the occlusion site is covered by the surface area of the implant 10. The implant 10 has a thickness T for the implant to reside within the pericardial space PS at the epicardial surface E.

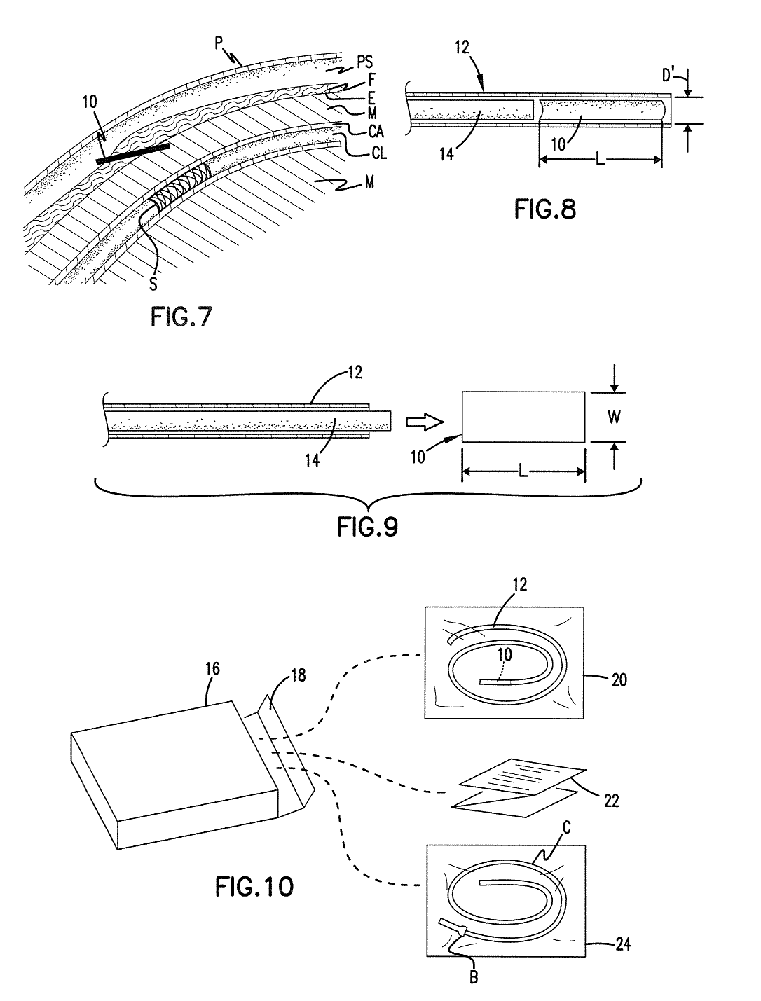

It will be appreciated that FIG. 5 shows a simplified version of anatomy. It does not show fat deposits which may reside on the epicardial surface E. Such is shown in FIG. 7 with a fat layer F shown on the epicardial surface E. The implant 10 is shown imbedded within the fat layer F and partially protruding through the epicardial surface E into the myocardium M. In the present invention, the implant 10 is placed at the epicardial surface with the term “at” meaning on or near the epicardial surface either spaced from the epicardial surface E by a fat layer F, directly on the epicardial surface E or imbedded within the myocardium M through the epicardial surface E.

The implant 10 is a carrier for a therapeutic agent to treat coronary disease residing beneath the position of the implant 10. In the embodiments of FIGS. 5 and 6, the drug is preferably an anti-restenosis drug such as those described under the heading “Therapeutic Agents” of this application. Such drugs may be surface coatings on the implant 10, impregnated within the material of the implant 10, or placed within holes formed within the implant 10 (all of which forms no part of this invention per se and may be as disclosed in the various documents described under “Therapeutic Agents”).

The implant 10 is delivered to the epicardial surface E through the pericardial space PS. Access to the pericardial space PS may be either surgical or percutaneous through any of the techniques disclosed in the previous section of this application titled “Pericardial Access Procedures”. In percutaneous access, implant 10 is preferably delivered through an atrial appendage into the pericardial space PS.

In either of the pericardial access procedures, a small diameter catheter 12 (FIG. 8) is admitted to the pericardial space PS for delivery of the implant 10. For pericardial access, such catheter 12 must be small diameter to permit passage through the patient's vasculature into the pericardial space PS while minimizing blood loss from the heart into the pericardial space PS. Further, in the less invasive surgical procedures, small diameter catheters are placed in the pericardial space to reduce the amount of incision through the pericardium P. As a consequence, the implant 10 has a first geometry (FIG. 8) for delivery to the pericardial space PS and a second geometry (FIG. 9) following discharge from a delivery device 12 into the pericardial space PS.

In FIG. 8, the implant 10 is shown rolled up in a coiled configuration at a distal end of a catheter 12 with the implant 10 presenting a diameter D to be fully positioned within the catheter 12. A push rod 14 is positioned within the catheter 12 just proximal to the implant 10. Relative movement of the catheter 12 and a push rod 14 (i.e., either distal movement of the push rod 14 or proximal movement of the catheter 12) ejects the implant 10 from the distal end of the catheter 12 as illustrated in FIG. 9. Upon discharge, the implant 10 assumes a planar configuration to overly the treatment area. Following such delivery, the delivery device 12 is removed leaving the implant 10 fully implanted within the patient and with no portion of the implant 10 exposed outside of the pericardial space PS.

The material of the implant 10 is biocompatible for chronic placement in the pericardial space PS. It may be an elastic material or material which assumes the second geometry by reason of phase change of the material. It will be appreciated that such materials (both plastic and metal) are well known in the art (such as nitinol and polymer materials). The therapeutic agent may be in a polymer coating on the material of the implant. The agent is selected that upon release it penetrates heart tissue to a coronary artery (e.g., capable of tissue penetration up to 5 mm). Such drugs are none in the art are in included in those described in the section “Therapeutic Agents”.

The implant 10 may contain radiopaque markers to permit visualization by a physician when placing the implant 10 within the pericardial space PS to ensure positioning over the stent S. The implant 10 may be placed during the same procedure at the placement of the stent S or may be placed before or after placement of the stent S. Drugs from the implant 10 are released over time and migrate through the myocardium M to the coronary artery at the site of the stent S to provide the desired therapy of anti-restenosis drugs at the stent site. Implant 10 may include sufficient drugs for full release (e.g., 90% of the original amount of the drug) of the drugs over a time period (for example, 30 to 45 days) to abate restenosis at the site of the stent S.

FIG. 10 illustrates a kit according to the present invention including a container 16 in the form of a cardboard box having a lid 18 for access to an interior of the container 16. The catheter 12 of the present invention is contained within a sterile pouch 20. Preferably, the sterile pouch 20 is a sealed plastic pouch of clear plastic to permit inspection of the catheter 12 through the pouch. The pouch 20 is contained within the container 16. Also contained within the container 16 are written instructions 22 for use of the catheter 12. Such instructions include instruction for placement of a distal end of the catheter 12 in the pericardial space PS and ejecting the implant 10 from the distal end to the epicardial surface E at the site of the obstruction O. Optionally, a container 16 may include a second sterile sealed pouch 24 containing devices for treatment of the occlusion such as a catheter C with a balloon B at a distal end.

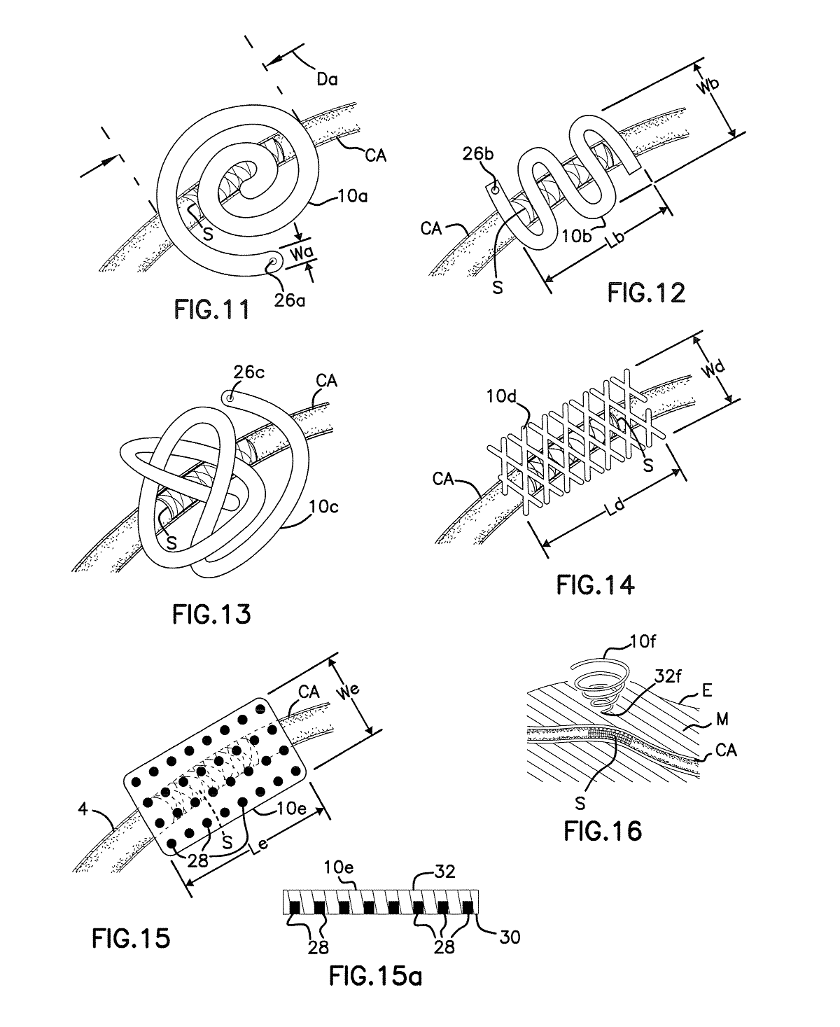

The previously described embodiment of the present invention is an implant 10 in the form of a implant sized to have a surface area overlying the epicardial surface E to cover the area of a stent S placed within a coronary artery CA beneath the epicardial surface E. FIGS. 11-15 show alternative embodiments of an implant for placement on the epicardial surface overlying a stent.

In FIGS. 11-15, the epicardial surface and the myocardium are not shown for ease of illustration. Also, the coronary artery CA is shown in longitudinal cross-section to reveal a prior art stent S at an occlusion site.

In FIG. 11, an implant 10a is shown in the form of a resilient coil. Within the delivery device 12, the coil 10a is deformed to a linear configuration (i.e., first geometry). The width Wa of the implant 10a is smaller than or equal to the diameter D of the catheter lumen. Following ejection of the implant 10a from the catheter 12, the implant 10a is biased to assume the coiled shape (i.e., second geometry) of FIG. 11 with the diameter Da greater than the width W′ of the coronary artery and the length L′ of the stent. An end of the implant 10a is provided with a hole 26 or other attachment location for later grabbing the end of the implant 10a and pulling it into a catheter for subsequent removal of the implant 10a from the epicardial surface.

In FIG. 12, an alternative embodiment is shown where the implant 10b may be straight and elongated (first geometry) within a catheter and assume a wavy configuration (second geometry) following discharge of the implant 10b. The length and width of the implant 10b in its second geometry (LB, WB) are preferably greater than the length and width L′, W′ of the area to be treated.

In FIG. 13, the implant 10c is, again, an elongated implant which, following discharge from a catheter 12 assumes a second geometry. In FIG. 13, the second geometry is a more random coiling and intertwining of the implant 10c. However, it continues to have the further benefit of the second geometry which is greater in overall surface area than the area occupied by the stent so that drugs eluted from the implant 10c cover the site to be treated.

In FIG. 14, the implant 10d has a construction similar to conventional stent construction in that it is a mesh. Different from cylindrical stents, the mesh of implant 10d is planar in configuration when in the second geometry having a length and width LD and WD greater than the length and width L′, W′. Mesh of 10d is compressible to fit within the diameter D of the catheter 12.

The implant 10e of FIG. 15 is similar to FIG. 14 in that the implant 10e is planar in configuration with a length and width Le, We greater than the length and width L′, W′ and with implant 10e compressible to fit within the diameter D of the catheter 12. The implant 10 has a plurality of holes 28 formed through a first surface 30 of the implant 10e and not projecting through the opposite second surface 32 (as best shown in the cross-sectional view of FIG. 15A. The holes 28 may be filled with the desired therapeutic agent. Such drug delivery is shown in U.S. Pat. No. 7,208,011 to Shanley et al. issued Apr. 24, 2007 (describing an implantable medical device with drug-filled holes). In previously described embodiments, either side of the implant may oppose the epicardial surface with drug delivery eluting from all exposed surfaces of the implants. In the embodiments of FIGS. 15 and 15A, the drug is delivered from the holes 28 only across the surface 30. No drugs are eluted across surface 32 into the pericardial space PS. The implant 10e is placed on the epicardial surface E with surface 30 opposing the epicardial surface E.

FIGS. 16-22 show embodiments of the present invention where implants carrying the therapeutic agent are placed wholly or partially into the myocardium M across the epicardial surface E from the pericardial space PS.

In FIG. 16, the implant 10f is a conical shaped coil and having a cone tip end 32f which is sharpened to penetrate into the myocardium M from the epicardial surface E. The base of the cone has an area greater than the area of the occlusion site.

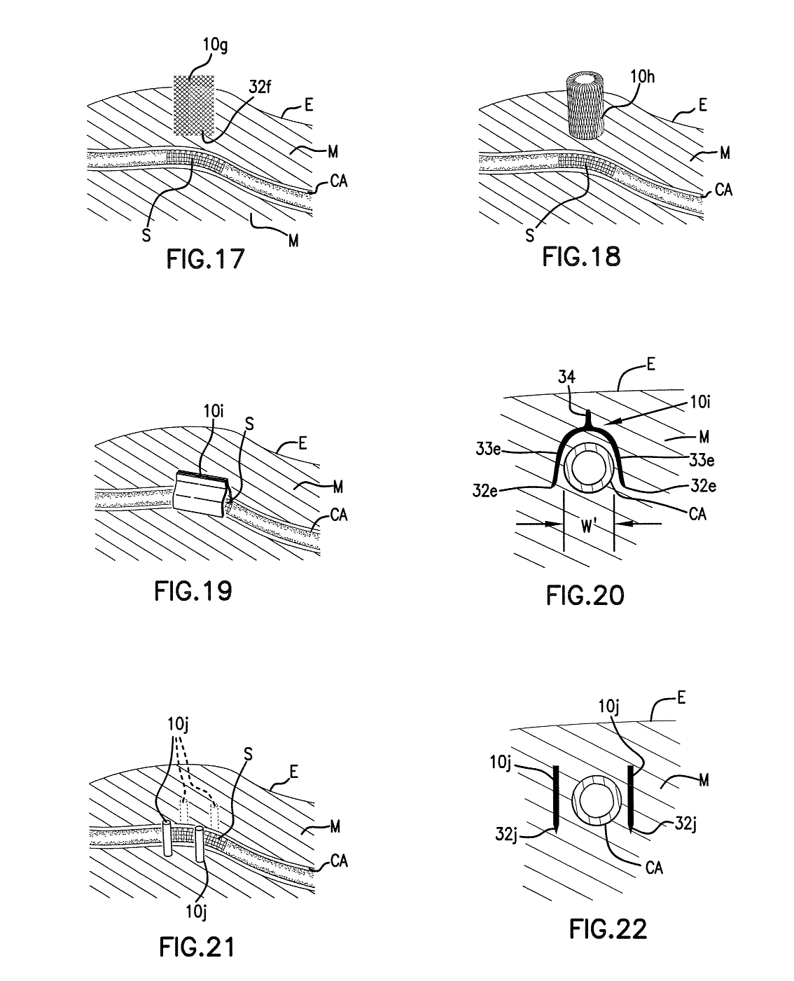

In FIG. 17, the implant 10g is a planar mesh construction having a sharp leading end 32f. In FIG. 18, the implant 10h is a cylindrical mesh construction which expands following ejection from the catheter 12. Within the catheter 12, the cylindrical implant 10h has a diameter less than or equal to diameter D. Following ejection, it expands to a greater diameter.

In FIGS. 19 and 20, the implant 10i is shown as a clip having side walls 33e which expand to be spaced apart greater than a width W′ of the coronary artery CA. Side walls 33e terminate at sharpened edges 32e to facilitate penetration from the epicardial surface E into the myocardium M with the coronary artery CA positioned between the side walls 33e. The side walls 33e are joined at a common handle 34 which may be grasped by any grasping tool passed through the catheter 12 to manipulate the implant 10i.

In FIGS. 21 and 22, the implant 10j is a plurality of rods containing a therapeutic agent for time delay delivery. The rods have sharpened distal ends 32j to permit placing a plurality of rods through the epicardial surface ES into the myocardium M with the rods positioned on opposite sides of the coronary artery CA.

In all of the disclosed embodiments, the implant discharges a therapeutic agent over time. The therapeutic agent migrates through the myocardial tissue to a desired treatment site of the heart. The implant is positioned within the heart at the epicardial surface by delivery from a delivery tool admitted into the pericardial space. Following delivery, the delivery tool is removed leaving the implant 10 completely contained within the heart (i.e., not exposed through the pericardium).

With the present invention now disclosed in the preferred embodiment, modifications and equivalents of the disclosed concepts may occur to one of ordinary skill in the art. It is intended that such modifications and equivalents be included within the scope of the claims which are appended hereto.

What is claimed is:

1. An apparatus for treating tissue of a heart of a patient, said apparatus comprising: a carrier having a therapeutic agent selected for treating cardiac tissue with said agent releasable from said carrier over a period of time following placement of said carrier at an epicardial surface of said heart.

2. An apparatus according to claim 1 wherein said carrier is contained within a delivery tool at a first geometry and changes to a second geometry following delivery of said carrier from said tool.

3. An apparatus according to claim 2 wherein said delivery tool is adapted for insertion of said carrier into a tissue of said heart from said epicardial surface.

4. An apparatus according to claim 2 wherein said delivery tool is adapted for placement of said carrier on said epicardial surface.

5. An apparatus according to claim 2 wherein said delivery tool is adapted for percutaneous placement of said distal end in said pericardial space.

6. An apparatus according to claim 2 wherein said delivery tool is adapted for surgical placement of said distal end in said pericardial space.

7. A method for treating tissue of a heart of a patient, said method comprising delivering a carrier to an epicardial surface of said heart with said carrier having a therapeutic agent selected for treating cardiac tissue with said agent releasable from said carrier over a period of time following placement of said carrier at said epicardial surface of said heart.

8. A method according to claim 7 wherein said carrier is inserted into tissue of said heart from said epicardial surface.

9. A method according to claim 7 wherein said carrier is placed against said epicardial surface.

10. A method according to claim 7 wherein said carrier is contained within a delivery tool at a first geometry and changes to a second geometry following delivery of said carrier from said tool.

11. A method according to claim 7 wherein said carrier is delivered to said epicardial surface through a percutaneous access to a pericardial space of said patient.

12. A method according to claim 7 wherein said carrier is delivered to said epicardial surface through a surgical access to a pericardial space of said patient.
